(19) United States
(12) Patent Application Publication
(10) Pub. No.: US 2013/0109927 A1
(43) Pub. Date: May 2, 2013

(54) SYSTEMS AND METHODS FOR REMOTE PATIENT MONITORING

(71) Applicant: Frank Menzel, Oakland, NJ (US)

(72) Inventor: Frank Menzel, Oakland, NJ (US)

(73) Assignee: MINDRAY DS USA, INC., Mahwah, NJ (US)

(21) Appl. No.: 13/284,304

(22) Filed: Oct. 28, 2011

(51) Int. Cl. A61B 5/00

(52) U.S. Cl. 600/300

(57) ABSTRACT

A system that includes a local patient monitor and a plurality of remote patient monitors. The local patient monitor may display a plurality of patient parameter waveforms and patient parameter numerical values for a local patient in a primary display area. The local patient monitor may further display patient parameter numerical values for a plurality of remote patients in a secondary display area. According to one embodiment, a medical practitioner may select one of the remote patients in order to display a plurality of patient parameter waveforms and patient parameter numerical values for a remotely located patient in the primary display area. According to one embodiment, a server may include a monitor communications module configured to identify remote patient data to be displayed on each of the plurality of patient monitors.

### TECHNICAL FIELD

This disclosure relates generally to patient monitors for monitoring a plurality of physiological parameters of a patient.

### BRIEF DESCRIPTION OF THE DRAWINGS

FIG. 1 illustrates a screen shot of one embodiment of a patient monitor interface that includes a primary display area and a secondary display area.

FIG. 2 illustrates a block diagram of one embodiment of a patient monitoring system, including a plurality of patient monitors and a server connected by a network.

FIG. 3 illustrates a flow chart of one embodiment of a method for displaying medical parameters of a local patient and for displaying medical parameters of a plurality of remote patients.

FIG. 4 is a flowchart of one embodiment of a method for remote patient monitoring that displays an alarm on each of a plurality of connected patent monitors when a specified physiological condition is detected by one of the patient monitors.

FIG. 5 is a perspective view of one embodiment of a patient monitor.

### DETAILED DESCRIPTION

Patient monitors may be used to analyze and display physiological parameters obtained from one or more sensors attached to a patient. The physiological parameters may include, for example, pulse, temperature, respiration, blood pressure, blood oxygen, electrocardiogram, etc. Physiological parameters of a patient may be displayed as a waveform or as a numerical value. A waveform may show the physiological parameter over a period of time, while a numerical value may show the present value of the physiological parameter. Patient monitors may be used by medical practitioners to monitor the condition of patients and to identify alarm conditions based upon monitored physiological parameters of a patient. Upon the detection of an alarm condition, an alarm may alert medical practitioners to the condition of a patient requiring immediate attention.

A medical practitioner is often responsible for the care of several patients, many of whom may be in different rooms or wards. Accordingly, it may be difficult or impossible for a single medical practitioner to constantly observe the condition of each patient. Patient monitors may be configured to sound an alarm when certain conditions occur; however, simply broadcasting alarms is not a complete solution. For example, the medical practitioner might not be aware that a remote patient monitor has malfunctioned or become disconnected and is no longer transmitting alarms. In addition, medical practitioners may be occupied with one patient for a period of time during which time it may be difficult for the medical practitioner to remain apprised of the condition of other patients in the care of the medical practitioner.

These and other problems may be addressed by the present disclosure. In one embodiment, a local patient monitor may display a plurality of physiological parameters for a local patient. In addition, the local patient monitor may display a remote monitoring interface along with the local patient's physiological parameters. The remote monitoring interface may include physiological parameters for a plurality of remote patients in addition to displaying physiological parameters for a local patient.

According to various embodiments, a primary display area may be provided in which a plurality of waveforms relating to a local patient may be displayed. A secondary display area may also be provided in which a plurality of numerical values relating to a group of remotely located patients may be displayed. According to various embodiments, a medical practitioner may be able to select one of the remotely located patients in order to display a plurality of patient parameter waveforms associated with the remotely located patient in the primary display area.

The embodiments of the disclosure will be best understood by reference to the drawings, wherein like elements are designated by like numerals throughout. In the following description, numerous specific details are provided for a thorough understanding of the embodiments described herein. However, those of skill in the art will recognize that one or more of the specific details may be omitted, or other methods, components, or materials may be used. In some cases, operations are not shown or described in detail in order to avoid obscuring more important aspects of the disclosure.

Furthermore, the described features, operations, or characteristics may be combined in any suitable manner in one or more embodiments. It will also be readily understood that the order of the steps or actions of the methods described in connection with the embodiments disclosed may be changed as would be apparent to those skilled in the art. Thus, any order in the drawings or detailed description is for illustrative purposes only and is not meant to imply a required order, unless specified to require an order.

Embodiments may include various steps, which may be embodied in machine-executable instructions to be executed by a general-purpose or special-purpose computer or other electronic device. Alternatively, the steps may be performed by hardware components that include specific logic for performing the steps or by a combination of hardware, software, and/or firmware.

Embodiments may also be provided as a computer program product including a computer-readable medium having stored thereon instructions that may be used to program a computer or other electronic device to perform the processes described herein. The computer-readable medium may include, but is not limited to: hard drives, floppy diskettes, optical disks, CD-ROMs, DVD-ROMs, ROMs, RAMs, EPROMs, EEPROMs, magnetic or optical cards, solid-state memory devices, or other types of media/computer-readable medium suitable for storing electronic instructions.

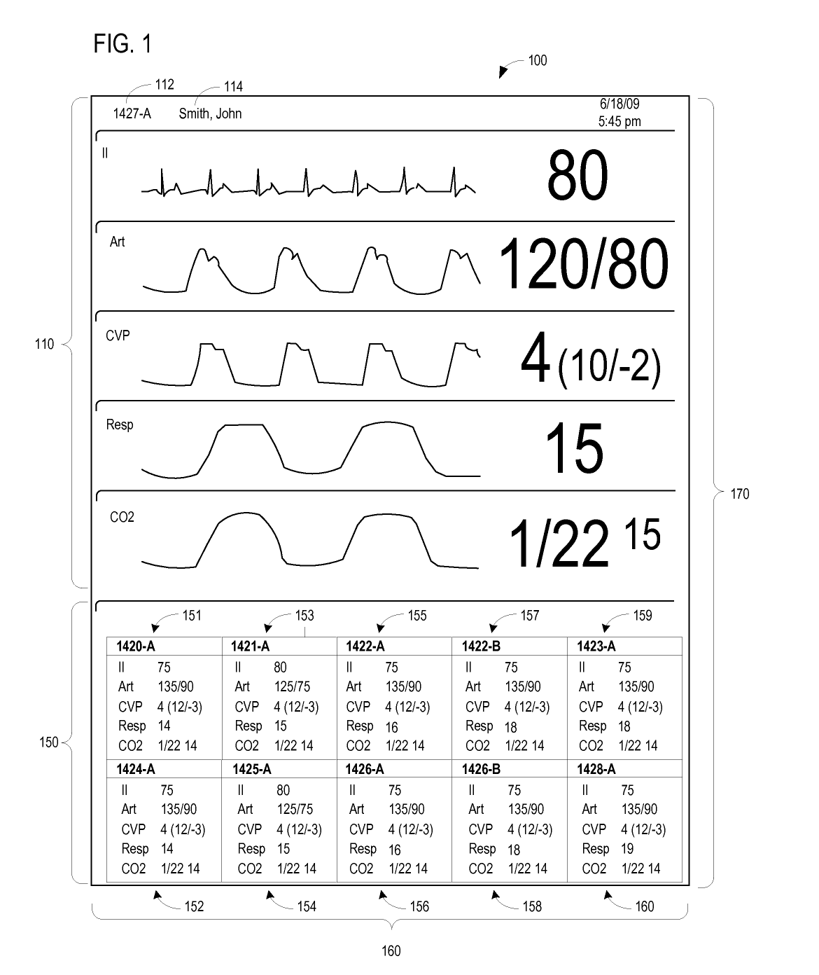

FIG. 1 illustrates a screen shot of one embodiment of a patient monitor interface 100. The patient monitor interface 100 includes a primary display area 110 and a secondary display area 150. The primary display area 110 may be configured to display a plurality of patient parameter waveforms and a corresponding plurality of patient parameters in a numerical format. The secondary display area 150 may be configured to display a plurality of patient parameters in a numerical format.

Secondary display area 150 includes a plurality of patient information display tiles 151-160, each of which displays a plurality of patient parameters in a numerical format. In order to protect patient privacy, patient information displayed in the secondary display area 150 may only be identified using anonymous indicators, such as a room number or a bed number. Patient privacy may be a concern, since the patient physiological parameters may be displayed in a number of other patient's rooms. In contrast, the primary display area 110 may include a patient's name 114 or other personally identifying information in addition to a bed or room number 112.

According to various embodiments, a patient monitoring system operable to generate the patient monitor interface 100 may be configured to monitor a plurality of physiological parameters of a local patient and a plurality of remotely located patients. Specifically, the physiological parameters may comprise one or more of a patient pulse, temperature, respiration, blood pressure, blood oxygen level, and electrocardiogram. According to alternative embodiments, more or fewer physiological parameters may be monitored or displayed by a patient monitor.

As illustrated in FIG. 1, the patient monitor interface 100 has a vertical dimension 170 that is larger than a horizontal dimension 160. According to various embodiments, the patient monitor interface 100 may be displayed on a commercially available display device having an aspect ratio of 16:9. The display device may be utilized in a portrait orientation. According to alternative embodiments, a display device having another aspect ratio may also be utilized. For example, a display device having an aspect ratio of 4:3 may be utilized. In such an embodiment, a secondary display area may be disposed, for example, to the side of a primary display area.

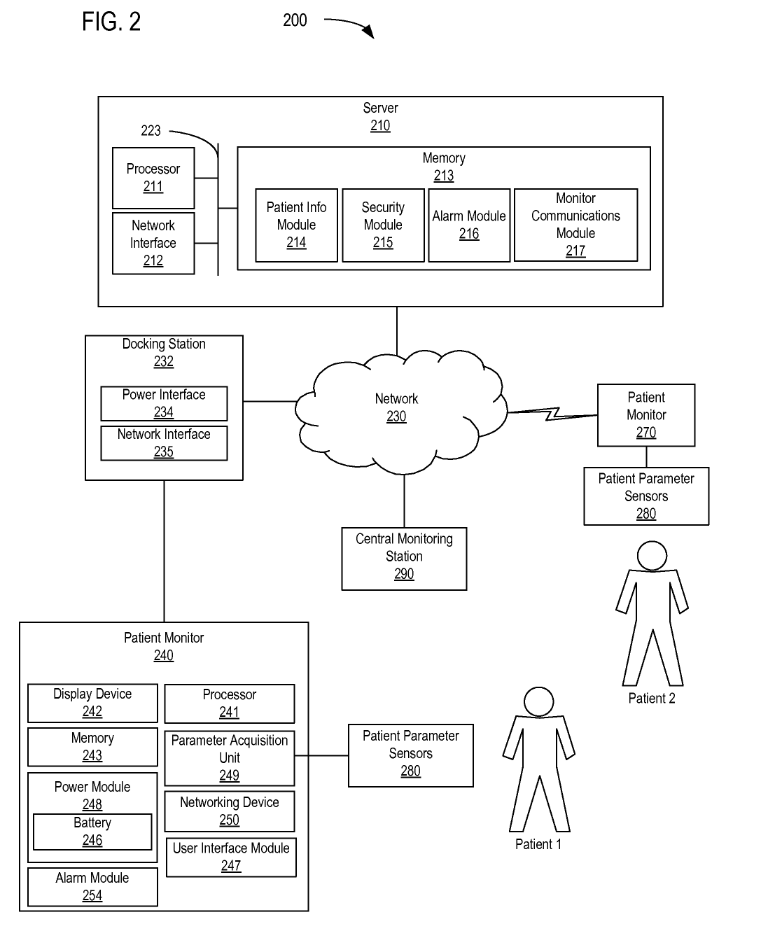

FIG. 2 illustrates a block diagram of a patient monitoring system 200 according to one embodiment. The patient monitoring system 200 includes a plurality of patient monitors 240 and 270, which analyze and display physiological parameters of patient 1 and patient 2, respectively. The patient monitors 240 and 270 may be configured to communicate through a network 230, such as a hospital's local area network (LAN) or the Internet.

In certain embodiments, a patient monitor (e.g., patient monitor 240) may be connected to the network 230 through a docking station 232. The docking station 232 may allow a patient monitor to be removed and transported between different locations in a hospital or other medical facility. In the embodiment illustrated in FIG. 2, the patient monitors 240 and 270 may be configured to be selectively coupled with, and selectively decoupled from, the docking station 232.

For illustrative purposes, the patient monitor 240 is shown as being coupled to docking station 232. In certain embodiments, the docking station 232 provides the respective patient monitor 240 with power and/or a connection to the network 230. Accordingly, the docking station 232 is illustrated as including a power interface 234 and a network interface 235. The power interface 234 may be configured to convert an alternating current (AC) power signal to a direct current (DC) power signal and/or provide power signal conditioning for the coupled patient monitor 240. The network interface 235 may include, for example, an Ethernet communication controller to allow the coupled patient monitor 240 to communicate to the network 230 through the docking station 232.

The patient monitor 240, according to the illustrated embodiment, includes a processor 241, a display device 242, a memory 243, a networking device 250, a power module 248, a parameter acquisition unit 249, a user interface module 247, and an alarm module 254. The processor 241 is configured to process patient data signals received through the parameter acquisition unit 249 and to display the patient data signals (e.g., as waveforms and/or numerical values) on the display device 242. The parameter acquisition unit 249 receives the patient data signals from one or more patient parameter sensors 280. The parameter acquisition unit 249 may be configured to process the acquired patient data signals in cooperation with the processor 241. The patient monitor 240 may store the patient data signals in the memory 243 along with other data. For example, the patient monitor 240 may store a current set of configuration settings in the memory 243.

According to the illustrated embodiment, patient monitor 240 is configured to communicate with the network 230 through the network interface 235 of the docking station 232, while patient monitor 270 is configured to wirelessly connect to the network 230. The networking device 250 may be configured to determine whether the patient monitor 240 is connected to the docking station 232, and whether the patient monitor 240 may utilize the network interface 235 of the docking station 232, or whether a wireless connection should be established to the network 230.

The power module 248 receives a power signal from the power interface 234 of the docking station 232. The power module 248 provides any necessary power conversions and distributes power throughout the patient monitor 240. The power module 248 may include a battery 246 that is charged through the power interface 234 while the patient monitor 240 is coupled to the docking station 232.

The user interface module 247, in cooperation with the processor 241 and the display device 242, may be configured to process and format the acquired physiological parameters associated with the patient for display in a graphical user interface (GUI). The user interface module 247 may also be configured to display physiological parameters associated with patient 2 that are received from another monitor (e.g., patient monitor 270).

An alarm module 254 may be configured to detect an alarm condition of a local patient. Upon the detection of an alarm condition, alarm module 254 may be configured to communicate alarm information to other patient monitors. Upon receipt of the alarm information, patient monitor 270 may display the alarm information in order to alert medical practitioners that patient 1 may require immediate attention.

A server 210 may be connected to network 230 via a network interface 212. The server 210 may be configured to facilitate the communication of patient data within system 200. The server 210 comprises a processor 211, a memory 213, and a network interface 212. A data bus 223 may provide a communication link between the processor 211, the memory 213, and the network interface 212. The processor 211 may operate using any number of processing rates, and/or architectures, and may be implemented using a general purpose or application specific processor. The processor 211 may be configured to perform various algorithms and calculations described herein. The processor 211 may be embodied as a general purpose integrated circuit, an application specific integrated circuit, a field-programmable gate array, and other programmable logic devices. The illustrated modules (reference nos. 214, 215, 216, and 217) are executable by the processor 211.

The memory 213 may be implemented using a variety of computer-readable storage media, including hard drives, RAM, solid-state memory devices, and other storage media suitable for storing electronic instructions and other data. Certain embodiments may be provided as a computer program product, including a computer-readable storage medium having stored instructions thereon that may be used to program a computer (or other electronic device) to perform processes described herein.

Although the embodiment illustrated in FIG. 2 illustrates various software modules located in the memory 213, it is contemplated that in other embodiments, the functions associated with the various software modules may be performed in other ways. For example, various subsystems may be employed that utilize application specific integrated circuits or other hardware implementations to perform the described functions. Embodiments employing a combination of both hardware and software configured to perform the functionality of the various modules are also contemplated. Further, the functions of various modules illustrated in FIG. 2 may be distributed throughout the system 200. Alternate embodiments may also include additional servers, which may operate as a distributed architecture.

The patient information module 214 may be configured to store patient information and pair that information with information regarding other patients in the care of the same group of medical practitioners. In other words, the monitor communications module 217 may identify which patient data is displayed on each patient monitor in order to maximize the availability of patient information for medical practitioners. For example, in a large hospital ward, multiple teams of medical practitioners may work simultaneously. Accordingly, it may be helpful to customize system 200 to display information about patients who are in the care of the same group of medical practitioners in specified rooms or areas that correspond to those patients in the care of the group of medical practitioners. The monitor communication module 217 may allow for patient information to be displayed in order to maximize the availability of patient data by displaying the data in the rooms of other patients in the care of the same group of medical practitioners.

A security module 215 may be configured to maintain the confidentiality of patient information. For example, the security module 215 may be configured to format information that is displayed in another patient's room so as to display only anonymous information. In addition, security module 215 may be configured to seek authentication from a user who seeks to display additional information of a remotely located patient.

An alarm module 216 may be configured for setting alarm conditions that may be detected by the patient monitor 240 or patient monitor 270. In one embodiment, a medical practitioner may specify safe ranges for various physiological parameters, outside of which an alarm should be triggered. For instance, the user may specify that an alarm should be triggered if a patient's systolic pressure exceeds 180 or drops below 80, or if the patient's diastolic pressure is greater than 100 or less than 50. For certain parameters, the term “range” may be represented as a single value, such as an upper or lower limit. Some alarms may be triggered by a combination of parameters being within particular ranges and/or exceeding or being lower than particular thresholds. When one of the patient monitors (e.g., patient monitor 240) detects an alarm condition, alarm information may be transmitted to one or more connected patient monitors (e.g., patient monitor 270). Alarm module 216 may facilitate the communication of alarm information from patient monitor 240 to patient monitor 270.

The monitor communications module 217 may be configured to facilitate communication among patient monitors 240 and 270. For example, monitor communications module 217 may format information for display on other patient monitors. The monitor communications module 217 may also facilitate communication of patient data to other systems, such as a central monitoring station 290. To the extent that conversion of patient data is needed for use of the patient data by multiple systems, the monitor communications module 217 may be configured to perform the conversion.

The central monitoring station 290 may also be in communication with the network 230. The central monitoring station 290 may display patient information for the patients in a hospital ward. The central monitoring station 290 may be located, for example, at a central nursing station.

According to some embodiments, a patient monitoring system may be implemented using a peer-to-peer architecture, rather than the client-server architecture, as shown in FIG. 2. In a peer-to-peer embodiment, the functions performed by the server 210 may be implemented by one or more patient monitors.

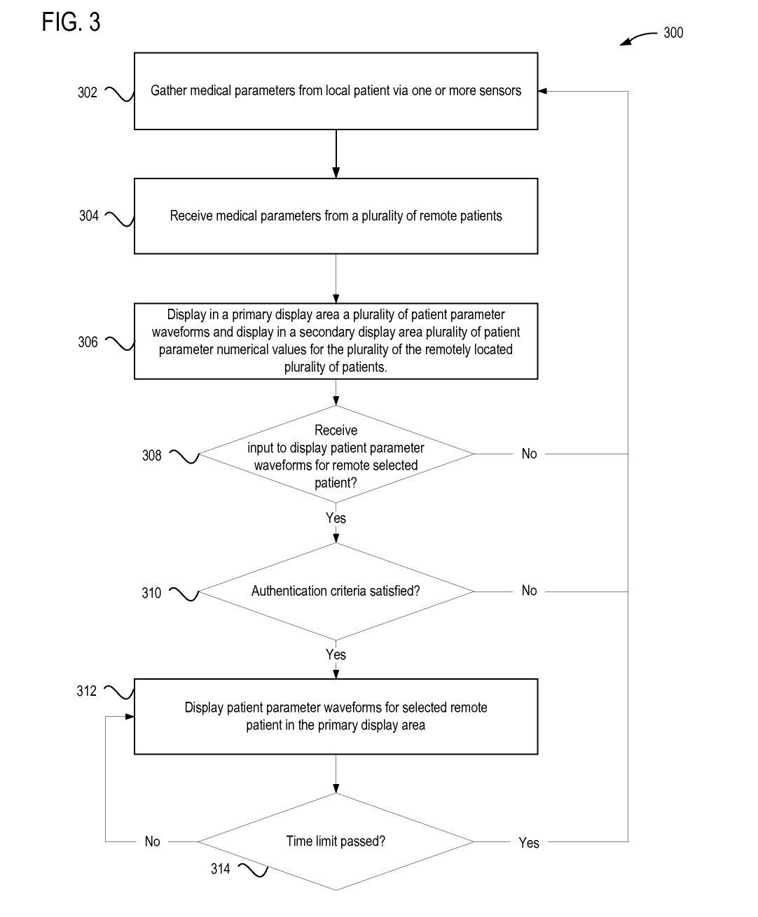

FIG. 3 illustrates a flow chart of a method 300 for displaying medical parameters of a local patient and for displaying medical parameters of a plurality of remote patients. At 302, medical parameters from a local patient are gathered via one or more sensors. For example, a patient monitor may monitor a patient's pulse, temperature, respiration, blood pressure, blood oxygen level, and electrocardiogram. Medical parameters from a plurality of remote patients may be received at 304. Such parameters may be received from a server that is configured to interface with a plurality of patient monitors, or such parameters may be received directly from a plurality of patient monitors.

A patient monitoring system implementing the method 300 may display medical parameters of the local patient in a primary display area and may display medical parameters of the reality of remote patients in a secondary display area, at 306. FIG. 1 illustrates one embodiment having a primary display area and a secondary display area. As illustrated in FIG. 1, patient parameter waveforms are displayed in the primary display area and numerical patient parameters are displayed in the secondary display area regarding other patients. A medical practitioner may select a patient whose information is displayed in the secondary display area and have that patient's information be displayed in the primary display area. At 308, it may be determined if input from a medical practitioner has been received to display a remote patient's parameters in the primary display area. If so, method 300 may move to 310. If not, method 300 may return to 302.

After receiving input from a medical practitioner to display a selected remote patient's parameters in the primary display area, the medical practitioner may be required to satisfy authentication criteria, at 310. The authentication criteria may include a password, PIN number, biometric authentication, or other forms of authentication. The authentication criteria may help to ensure that remote patient information is only available to medical practitioners who are caring for the patient, and thus, can provide appropriate authentication credentials. If the authentication criteria are not satisfied, method 300 may return to 302, and method 300 may continue without displaying patient parameters for the selected remote patient in the primary display area. If the authentication criteria are satisfied, at 312, patient parameter waveforms for the selected remote patient may be displayed in the primary display area. A time limit may be specified that limits the amount of time that patient parameters for the selected remote patient are displayed in the primary display area. At 314, it may be determined whether the time limit has passed. If so, method 300 may return to 302. If not, method 300 may return to 312.

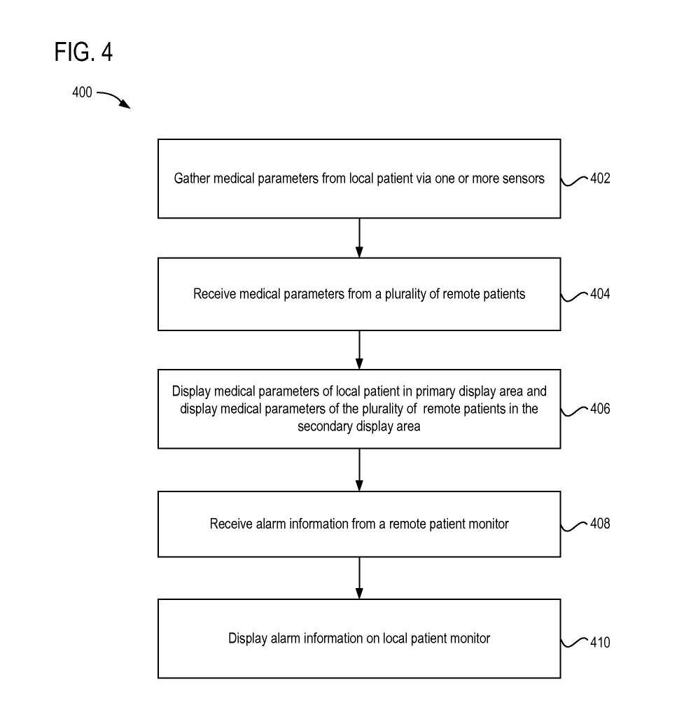

FIG. 4 is a flowchart of one embodiment of a method 400 for remote patient monitoring that displays an alarm on each of a plurality of connected patient monitors when a specified physiological condition is detected by one of the patient monitors. At 402, medical parameters from a local patient are gathered via one or more sensors. At 404, the patient monitor may obtain a remote monitoring status from each of a plurality of remote patient monitors. The patient monitor may then display the medical parameters of the local patient in a primary display area and display medical parameters of the plurality of remote patients in the secondary display area, at 406.

At 408, a patient monitor implementing method 400 may receive alarm information from a remote patient monitor indicating that one or more physiological parameters for a remote patient are outside of a determined range. The alarm information may include location information (e.g., bed or room number), as well as an indication of an alarm condition (e.g., tachycardia). At 410, the patient monitor may display the alarm information on the local patient monitor.

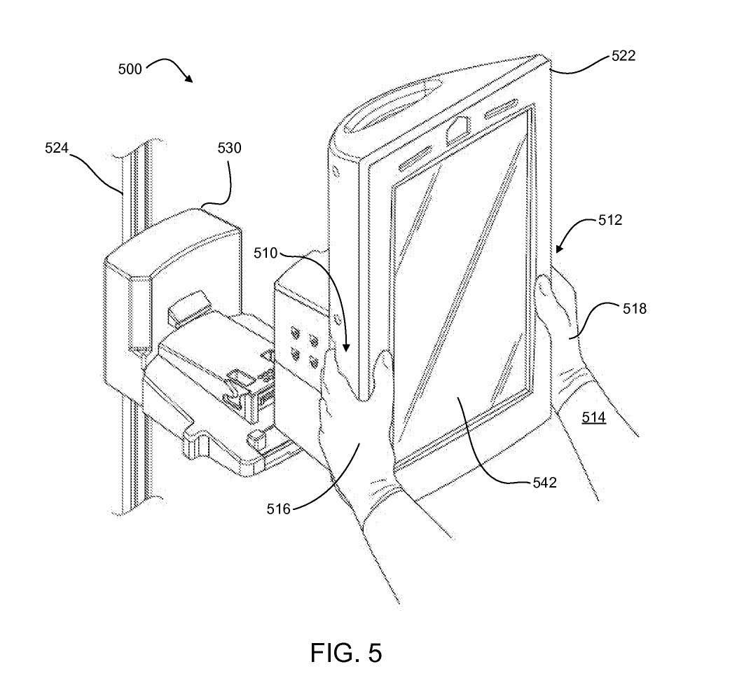

FIG. 5 is a perspective view of a patient monitor 500, according to one embodiment. The patient monitor 500 includes a patient monitor 522 and a docking station 530. The patient monitor 522 can be configured to selectively couple with and decouple from the docking station 530. The coupling between the patient monitor 522 and the docking station 530 can be mechanical, electrical, optical, and/or any other suitable variety. For example, the coupling can be for physical union, power transfer, and/or communication.

The patient monitor 522 may include one or more gripping regions 510, 512 that are configured to aid in coupling and decoupling the patient monitor 522 from the docking station 530. For example, a medical practitioner 514 can firmly grasp with his or her hands 516, 518 the gripping regions 510, 512 during removal of the patient monitor 522 from the docking station 530. When the patient monitor 522 is separated from the docking station 530, the full weight of the patient monitor 522 can be supported by a grip of the medical practitioner 514 on the gripping regions 510, 512.

The patient monitor 500 may include one or more actuators (not shown) which, when actuated, permit release of the patient monitor 522 from the docking station 530. The actuators can be integrated into the gripping regions 510, 512 or other portions of the patient monitor 522, so as to permit for convenient and continuous-movement dismounting of the patient monitor 522. For example, in some embodiments, a practitioner 514 can actuate an actuator using a hand 516, 518 while that hand 516, 518 is simultaneously holding a respective gripping region 510, 512.

In FIG. 5, the patient monitor 522 is illustrated as having been removed from the docking station 530. A front surface of the patient monitor 522 can include a display device 542 that is configured to display information in a visually perceivable format. The display device 542 may be of any suitable variety, including those presently known and those yet to be devised. For example, the display device 542 may include a liquid crystal display (LCD) panel. In some embodiments, the display device 542 may be configured to receive information or otherwise interact with a medical practitioner. For example, the display device 542 may include a touch screen.

The patient monitor 522 may include one or more ports for receiving or delivering information, which can include one or more serial ports, USB ports, Ethernet ports, DVI ports, or any other suitable variety of ports, interfaces, or connectors. In some embodiments, information received via one or more of the ports can be displayed on the display device 542.

At least a portion of the information displayed by the patient monitor 522 may represent information received from a patient or that otherwise relates to the patient. For example, in some embodiments, one or more sensors (not shown) are connected to the patient to sense a particular parameter, and information obtained via the one or more sensors. The sensors may deliver information to the patient monitor 522 via one or more cables (not shown) connected to one or more ports.

The patient monitor 522 may be configured to both mechanically and electrically couple with the docking station 530. The patient monitor 522 may receive power from the docking station 530, which itself can receive power from a power source (not shown) via a power line or cord. The power source may include, for example, the AC wiring of a hospital.

The docking station 530 may be mounted in a substantially fixed position. For example, the docking station 530 may be rigidly mounted to a wall within a hospital room in a single position by one or more plates, brackets, screws, bolts, or other mounting hardware and attachment devices. As another example, the docking station 530 may be configured to transition among multiple fixed positions. For example, in the illustrated embodiment, the docking station 530 is coupled to a mounting strip 524, which is in turn mounted to a wall (not shown) of a hospital room. The docking station 530 is capable of being adjusted upward or downward along a path constrained by one or more channels defined by the mounting strip 524 so as to transition among a variety of positions. In each such position, the docking station 530 can be fixed relative to the mounting strip 524. In some embodiments, the docking station 530 is coupled with the mounting strip 524 via a mounting plate or a mounting bracket (not shown), the position of which can be adjusted upward or downward within the channels in any suitable manner.

In other embodiments, the docking station 530 may be secured to a hospital bed (not shown), a mechanical arm (not shown), or any other suitable object. In some embodiments, a bottom surface of the docking station 530 is positioned at a height of from about five feet to about six feet above a floor of a hospital room, so as to allow the patient monitor 522 to be viewed easily and/or to avoid interference with other objects in the room.

It will be understood by those having skill in the art that many changes may be made to the details of the above-described embodiments without departing from the underlying principles of the invention. The scope of the present invention should, therefore, be determined only by the following claims.

1. A patient monitor, comprising:
a parameter acquisition unit configured to acquire a plurality of data signals corresponding to a plurality of physiological parameters of a local patient;
a processing unit communicatively coupled with the parameter acquisition unit configured to process the plurality of data signals and to generate patient parameter information related to the plurality of physiological parameters based on the plurality of data signals, the parameter information comprising a plurality of local patient parameter waveforms and a plurality of local patient parameter numerical values;
a networking unit communicatively coupled with the processing unit and configured to receive a plurality of remote patient parameters relating to a plurality of remotely located patients;
a display device configured to:
display in a primary display area the plurality of local patient parameter waveforms; and
display in a secondary display area the plurality of remote patient parameters.

2. The patient monitor of claim 1, wherein the display device comprises a display area having a vertical dimension that is larger than a horizontal dimension.

3. The patient monitor of claim 2, wherein a ratio of the vertical dimension to the horizontal dimension is 16:9.

4. The patient monitor of claim 1, wherein the one or more physiological parameters comprise one or more of a patient pulse, temperature, respiration, blood pressure, blood oxygen level, and electrocardiogram.

5. The patient monitor of claim 1, wherein the display device is further configured to visually display remote patient parameter information relating to one or more physiological parameters of a remote patient in a remote patient region of the patient parameter interface.

6. The patient monitor of claim 1, wherein the display device is further configured to display in the primary display area numerical values associated with the plurality of parameter waveforms.

7. The patient monitor of claim 1, wherein the display device comprises a touch screen display.

8. The patient monitor of claim 1, wherein the display device is further configured to selectively display the plurality of patient parameter waveforms of a selected remotely located patient.

9. The patient monitor of claim 1, wherein the patient monitor is configured to dock with a docking station.

10. The patient monitor of claim 9, further comprising a battery configured to charge when docked with the docking station.

11. The patient monitor of claim 1, wherein the networking unit is configured for wireless communication.

12. The patient monitor of claim 1, wherein the display device is further configured to display an alarm in response to receiving alarm information via the networking unit.

13. A method for monitoring a plurality of patients using a plurality of patient monitors, the method comprising:
gathering medical parameters for a local patient via a sensor;
receiving medical parameters relating to a plurality of remote patients;
displaying a plurality of patient parameter waveforms gathered via the sensor in a primary display area;
displaying a plurality of remote patient parameter numerical values of the plurality of remote patients in a secondary display area;
receiving a selection of one of the plurality of remote patients; and
displaying patient parameter waveforms of the selected remotely located patient in the primary display area.

14. The method of claim 13, further comprising:
receiving authentication criteria prior to displaying patient parameter waveforms of one of the remotely located patients in the primary display area.

15. The method of claim 13, further comprising:
determining that a time line has passed; and
resuming the display of the plurality of patient parameter waveforms gathered via the sensor in a primary display area.

16. The method of claim 13, further comprising:
receiving alarm information from a remote patient monitor; and
displaying the alarm information.

17. A system comprising:
a network;
a plurality of patient monitors in communication with the network, each of the plurality of patient monitors configured to monitor a patient and each of the plurality of patient monitors comprising:
a parameter acquisition unit configured to acquire a plurality of data signals corresponding to a plurality of physiological parameters of a local patient;
a processing unit communicatively coupled with the parameter acquisition unit configured to process the plurality of data signals and to generate patient parameter information related to the plurality of physiological parameters based on the plurality of data signals, the parameter information comprising a plurality of local patient parameter waveforms and a plurality of local patient parameter numerical values;
a networking unit communicatively coupled with the processing unit and configured to receive a plurality of remote patient parameters relating to a plurality of remotely located patients;
a display device configured to:
display in a primary display area the plurality of local patient parameter waveforms; and
display in a secondary display area the plurality of remote patient parameters; and
a server comprising:
a monitor communications module configured to identify remote patient data to be displayed on each of the plurality of patient monitors.

18. The system of claim 17, wherein the server further comprises:
a security module configured to obtain authentication in response to input to display in the primary display area a plurality of remote patient parameter waveforms.

19. The system of claim 17, wherein the server further comprises:
an alarm module configured to receive an alarm condition from one of the plurality of patient monitors and to transmit alarm information to each of the other plurality of patient monitors.

20. The system of claim 17, wherein the server further comprises:
a patient information module configured to store patient information and to coordinate with the monitor communications module to display remote patient information on patient monitors located in patient rooms in the care of the same group of medical practitioners.
